(19) United States
(12) Patent Application Publication
(10) Pub. No.: US 2017/0209696 A1
(43) Pub. Date: Jul. 27, 2017

(54) SYSTEMS AND METHODS TO IMPROVE ANTI-TACHYCARDIAL PACING (ATP) ALGORITHMS

(71) Applicant: CardioFlow Technologies, LLC, Nashville, TN (US)

(72) Inventor: Daniel Walter Kaiser, Palo Alto, CA (US)

(21) Appl. No.: 15/423,560

(22) Filed: Feb. 2, 2017

Related U.S. Application Data
(63) Continuation-in-part of application No. 14/811,719, filed on Jul. 28, 2015.
(60) Provisional application No. 62/290,417, filed on Feb. 2, 2016.

(51) Int. Cl. A61N 1/362; A61N 1/365; A61B 5/0464

(52) U.S. Cl. CPC A61N 1/3622; A61B 5/0464; A61N 1/36514

(57) ABSTRACT

Apparatus, systems and methods are provided for prevention and/or remediation of cardiac arrhythmias, e.g., for optimizing anti-tachycardia pacing (ATP) algorithms. More particularly, implantable devices are provided that measure and treat cardiac arrhythmias. By monitoring the ATP attempt from additional electrodes, far-field morphology analyses, and/or measuring the return interval from a failed ATP attempt, the devices may estimate when entrainment has occurred, the amount of delay within the reentrant tachycardia, and/or tachycardia termination/acceleration. These variables and occurrences can be used to optimize the first and/or subsequent ATP attempts. Furthermore, other exemplary embodiments describe methods to integrate electrical restitution properties into the design of ATP pacing algorithms to facilitate tachycardia termination.

### RELATED APPLICATION DATA

The present application claims benefit of co-pending provisional application Ser. No. 62/290,417, filed Feb. 2, 2016, and is a continuation-in-part of co-pending application Ser. No. 14/811,719, filed Jul. 28, 2015, the entire disclosures of which are expressly incorporated by reference herein.

### FIELD OF THE INVENTION

This invention is in the field of devices and methods to diagnose and treat cardiac arrhythmias.

### BACKGROUND

Improper electrical activity inside of the heart can result in abnormally fast heart rates, called tachycardia. Depending on the rate and chambers of the heart involved (atria versus ventricles), these tachycardias can be symptomatic (palpitations) or life threatening. Implantable cardiac defibrillators (ICDs) are devices designed to treat these abnormal rhythms. In addition to delivering a powerful shock (defibrillation), these and similar implantable devices may deliver carefully timed pacing pulses (called anti-tachycardia pacing, or ATP) to the heart in order to terminate the tachycardia.

To terminate an arrhythmic circuit, a pacing stimulus is delivered from an electrode near or within the heart such that the resulting wave propagation interrupts and ultimately stops the reentrant tachycardia. In order to affect the tachycardia, the paced wavefront must reach the reentrant circuit (i.e., the region within the heart tissue causing the tachycardia). However, the initial pacing wavefronts may not reach the reentrant circuit, since these wavefronts typically collide with wavefronts leaving the tachycardia. With continuous pacing, the paced wavefront reaches part of the reentrant circuit (the “entrance site”) earlier than the native wavefront would have reached this aspect of the circuit. This wavefront advances the tachycardia. The paced wavefront also collides with the native tachycardia along the reentrant circuit. If the wavefront reaches an aspect of the reentrant circuit when this myocardial tissue has not repolarized, the wavefront may terminate. In other cases, the wavefront may reach myocardial tissue that conducts the wavefront; however, the wavefront velocity significantly decreases.

In most cases, in order to maintain a reentrant tachycardia, there is an area of myocardium with delayed conduction velocity. This area is typically within a critical isthmus. If the paced wavefront reaches the critical isthmus before this tissue has had enough time to repolarize, the tachycardia will terminate. In other cases, the paced wavefront may accelerate the tachycardia, although the critical isthmus has repolarized. In these cases, the conduction velocity may significantly delay. The delay in conduction velocity “protects” more distal aspects of the reentrant circuit from experiencing the total prematurity of the paced stimulations. Therefore, several pacing stimulations, after reaching the reentrant circuit, may be required to terminate the tachycardia.

Numerous different pacing modalities and algorithms have been created for the termination of tachycardia. These algorithms have been created for both atrial and ventricular tachycardias. These algorithms are programmed into implanted devices such as a pacemaker or implantable cardioverter-defibrillators (ICDs). These devices may deliver a high powered electrical shock, which attempts to reset all cells involved in the reentrant tachycardia in order to terminate the tachycardia. These shocks are often painful and can cause harm to myocardial cells. Alternatively, the devices may deliver anti-tachycardia pacing (ATP), whereby paced wavefronts reach critical aspects of the reentrant circuit in such a manner that the tachycardia terminates. ATP is usually painless and therefore has advantages over high-powered cardioversions.

ATP is not always successful at terminating the tachycardia. In this circumstance, the ATP is repeated at the same or different pacing algorithm in attempts to terminate the arrhythmic into a normal sinus rhythm. If ATP is unsuccessful, the patient may require high voltage cardioversion. ATP is unsuccessful in approximately 10-40%/o of ATP attempts. In addition, ATP sometimes accelerates the rhythm to a faster rate or may degenerate the rhythm into ventricular fibrillation, which is a chaotic rhythm that is not capable of sustaining life. Furthermore, since ventricular tachycardia frequently pumps less blood than the body requires, ventricular tachycardia may precipitate ventricular fibrillation or result in the patient passing out (syncope).

Thus, improved methods are needed for increasing the success rate of ATP and for decreasing the time in tachyarrhythmia, which will reduce the need for painful ICD shocks.

The efficacy of delivering anti-tachycardia pacing (ATP) through the right ventricular implantable cardioverter defibrillator (ICD) lead to terminate life-threatening fast ventricular tachycardia (FVT) was first published in 2001 by Wathen et al. In this study, the authors revealed that ATP could prevent ICD shock delivery in three of four episodes. Over the following years, ATP has become a valuable option to treat most VT episodes. Large-scale studies, including PainFree Rx II, EMPIRIC, PREPARE, or ATPonFastVT, have demonstrated the efficacy and safety of this approach. Moreover, delivering ATP instead of defibrillation has dramatically reduced the number of painful ICD shocks.

Typically, the ATP algorithm depends on the type (atrial versus ventricular) and rate of the tachycardia. For example, most device makers make a distinction between ventricular tachycardia (VT), fast ventricular tachycardia (FVT), and ventricular fibrillation (VF) based on the rate of the tachycardia. The tachycardia rate can be described in terms of beats per minute (BPM) or can be thought of as the time between heart beats (termed the RR interval). This time is also termed the tachycardia cycle length (TCL), and is often given in milliseconds. 60,000 (the number of milliseconds in a minute) divided by the heart rate provides the cycle length in milliseconds (ms). For example, a tachycardia of 200 BPM has a tachycardia cycle length of 300 ms.

ATP can be a pacing train of a certain cycle length (burst pacing) or can shorten the cycle length with each additional paced beat (often termed ramp pacing). Ramp therapy consists of a decremental drive of a programmable number of pulses, starting at a rate proportional to the current tachycardial cycle length (TCL).

The current algorithms of ATP are well known and easily accessible. One example is by implantable device maker Medtronic, although all device makers have created similar pacing algorithms. In Medtronic's PainFree Rx II Trial, the first therapy in the FVT zone (188-250 BPM) was two ATP sequences (8-pulse burst pacing train at 88% of the FVT cycle length). If the first ATP sequence was unsuccessful, the second sequence was delivered at 88% of the FVT cycle length minus ten milliseconds (10 ms). ATP therapies were delivered at maximum voltage and pulse duration. Programming of subsequent FVT therapies was left to the investigators' discretion and usually involved ICD shocks.

A recent study by Martins et al published in Eurospace (2012) performed a study involving the major ICD device makers: Biotronik, Boston Scientific, Medtronic, St Jude Medical and Sorin Group companies. In this study, the ventricular ATP algorithm was as follows: Implantable cardioverter/defibrillators were programmed to deliver ten ATP attempts for FVT cycle lengths (CLs) of 250-300 ms (200-240 BPM) before shock delivery (five bursts, then five ramps; 8-10 extrastimuli at 81-88% FVT CL; minimal pacing CL 180 ms). A total of 1839 FVTs, 1713 of which were ATP-terminated (unadjusted efficacy ¼ 93.1%, adjusted ¼ 81.7%). Furthermore, over 20% of the patients experienced ATP that required more than two episodes of ATP. Thus, there is room to improve the current ATP algorithms to reduce the time spent in tachycardia and prevent ICD shock.

The most advanced pacemakers feature atrial preventive pacing and atrial anti-tachycardia pacing (DDDRP), which may reduce atrial fibrillation occurrence and duration. The pacemakers may automatically deliver ATP therapies when an episode is classified as atrial tachycardia, which may last longer than a programmable ‘time to first therapy’ (often one minute). Often, ramp is programmed in order to deliver three series often sequences each, so that each patient could receive up to thirty termination attempts. Each series begins with a train often pulses. The first pulse of each of the three series is delivered at 91, 84, and 81% of the underlying atrial tachycardia cycle length (ATCL), respectively. In each series, subsequent pulses were delivered with a decrement in pacing coupling interval often millisecond (10 ms) each. If a previous train fails to terminate AT, an additional stimulus is added to the next train.

Burst+ therapy uses a drive of a programmable number of atrial pulses, the rate of which is proportional to the current ATCL, followed by up to two extrastimuli. Burst+ is programmed in order to deliver three series often sequences each; each sequence is made up of fifteen pulses followed by two extrastimuli. As in the Ramp programming, each patient can receive up to thirty termination attempts. The first scan of each series is released at 84% of the underlying ATCL. The first extrastimulus is delivered at 81% of the underlying ATCL; the second extrastimulus was delivered with an interval reduced by twenty milliseconds (20 ms). In the event of failure, the ATP train coupling interval was decreased by ten millisecond (10 ms) for each subsequent scan.

For both therapies, the minimal pacing interval (MPI) was one hundred fifty milliseconds (150 ms), so that pulses programmed at a shorter pacing interval than the MPI were delivered at the MPI value. Atrial ATP was recently found to significantly reduce the progression of atrial tachycardia to permanent atrial fibrillation (61% relative risk reduction) over a two year follow-up.

Previous methods use similar strategies for all tachycardias. These strategies use a burst or a ramp strategy with variable number of beats. If this ATP attempt fails, another burst or ramp is delivered at shorter intervals (faster rates).

The present application provides systems and methods that use unique pacing algorithms to improve the ability of a device to terminate a tachycardia.

### SUMMARY

The present invention is directed to apparatus, systems and methods for prevention and/or remediation of cardiac arrhythmias, e.g., optimizing anti-tachycardia pacing (ATP) algorithms. More particularly, the present invention is directed to implantable devices that measure and treat cardiac arrhythmias. The present application describes apparatus, systems, and methods whereby the pacing electrode, timing intervals, and pacing output are adjusted in order to hasten tachycardia termination and/or optimize the probability of tachycardia termination.

In one exemplary embodiment, the far-field morphology and/or timing intervals between electrodes are utilized to determine the optimal electrode to deliver ATP therapy. In another example, the pacing output is adjusted during ATP in order to increase efficacy.

In one exemplary embodiment, the device may identify reentrant tachycardias based on timing intervals, far-field morphology, and/or the time differences between multiple electrodes. The device may also monitor for arrhythmia entrainment and/or termination during deliver of ATP to optimize the current and future ATP algorithms. By measuring dynamic responses occurring from “overdrive pacing” (i.e., pacing using any of the ATP algorithms and systems ad methods herein) from far-field morphology and/or timing intervals of cardiac signals from additional electrode(s), other embodiments herein may estimate and record certain aspects of the ATP attempt, such as conduction delays within certain aspects of the tachycardia or relative distances to the critical aspects of the reentrant circuit. Therefore, the following concepts are advantageous to programming and designing device ATP capabilities and algorithms.

In another embodiment, the time interval from the stimulation from a first or stimulating electrode to the sensed ventricular signal of at least one second or sensing electrode when the patient is not in a ventricular tachycardia is measured. This time represents the conduction time to travel from the stimulating electrode to the sensing electrode. If the reentrant circuit is located between these two electrodes, this time interval sensed during overdrive pacing can be used to guide the overdrive pacing algorithm. This is because during tachycardia entrainment, the time interval between the pacing pulse (aka pacing stimulation) and the sensed ventricular signal is longer during overdrive pacing when in a reentrant ventricular tachycardia compared to this time interval when not in a ventricular tachycardia. The depolarizing wavefront travels through the reentrant circuit, often through an area of slow conduction, prolonging the conduction time. The pathway of the depolarizing wavefront that occurs when not in tachycardia entrainment is blocked by the depolarizing wavefront leaving the exit site of the reentrant circuit. Therefore, this sensed electrogram (ventricular signal) from the second electrode can be used to determine tachycardia entrainment.

In addition, prolongation of this time interval with additional overdrive pacing (after tachycardia entrainment) can be credited to conduction delay within the reentrant circuit. Since this conduction delay will also delay the tachycardia cycle length, this measured conduction delay can be used to deliver longer ‘priming’ pulses or ‘long’ pacing pulses compared to the pacing cycle length used to obtain tachycardia entrainment while maintaining access to the entrance site of the reentrant circuit. In other words, a pacing pulse with a cycle length longer than the initial tachycardia cycle length can be delivered while still overdrive pacing the ventricular tachycardia.

Therefore, by analyzing the timing and morphology of the ventricular signal from the second electrode during overdrive pacing, the overdrive pacing algorithm can be adjusted to optimize tachycardia termination. Specifically, when overdrive pacing causes block of the orthodromic wavefront traveling within the reentrant circuit (typically within the area of slow conduction), the second electrode often does not sense a ventricular signal from the pacing pulse at all. All wavefronts traveling towards the second electrode either terminate after colliding with the wavefront that leaves from the exit site or blocks within the reentrant circuit. If the electrodes are positioned in an optimal location relative to the myocardial tissue and scar distribution, monitoring the ventricular signal from the second electrode can identify tachycardia termination prior to delivering the next pacing pulse.

Alternatively, the cycle length of the ‘priming’ or ‘long’ pacing pulse can be prolonged until sensing of a local depolarization from the second electrode. In other scenarios, depending on anatomy and electrode distribution, although tachycardia termination cannot be verified prior to delivering the next pacing pulse, the next pacing pulse will create a depolarizing wavefront that reaches the second electrode at the same time interval as recorded when the patient is not in a ventricular tachycardia. In other scenarios, the wavefront from the stimulating electrode will reach the second electrode before the wavefront that leaves the exit site of the reentrant circuit. In this situation, the second electrode will not be as helpful in guiding the overdrive pacing algorithm.

In another embodiment, adjusting the delivery of overdrive pacing includes both the timing of pacing pulses and the power output of the overdrive pacing. By delivering higher power, the pacing electrode captures a larger area of myocardium. This maneuver can be utilized to advance the reentrant tachycardia without requiring a shorter pacing cycle length. In some embodiments, the ‘short’ or ‘accelerating’ pacing pulse regularly uses a higher power output than the ‘long’ or ‘priming’ pacing pulses.

In another embodiment, adjusting the delivery of overdrive pacing includes alternating at least one ‘long’ pacing pulse with a pacing cycle length greater than 90% of the tachycardia cycle length followed by at least one ‘short’ pacing pulse with a pacing cycle length less than 90% of the tachycardia cycle length. The ‘long’ or ‘priming’ pacing pulse performs two key functions. First, it prolongs the repolarization rate of the critical isthmus within the reentrant circuit, favoring tachycardia termination with subsequent pacing pulses. Next, the ‘long’ or ‘priming’ pacing pulses provide an opportunity to analyze for tachycardia entrainment and/or termination.

In another embodiment, the pacing cycle length of the ‘short’ pacing pulses are gradually shortened during the overdrive pacing until there is evidence of either tachycardia termination or tachycardia acceleration of the ventricular tachycardia. Furthermore, by estimating the conduction delay within the reentrant circuit after the ‘short’ pacing pulses, the pacing cycle length of the ‘long’ pacing pulses can be prolonged. Therefore, the ‘short’ pacing pulses are gradually becoming shorter while the ‘long’ pacing pulses are gradually becoming longer. Both of these intervals are gradually changed in order to facilitate tachycardia termination.

Furthermore, depending on the slope of the restitution curve, simply alternating the cycle lengths of both the ‘long’ and ‘short’ pacing pulses can result in tachycardia termination. In other words, a steep restitution curve of the myocardial tissue within the critical isthmus can facilitate the creation of an unstable harmonic that results in tachycardia termination. Similar to pushing a swing, small changes in pacing cycle lengths can lead to large swings in action potential duration that facilitate tachycardia termination.

In another embodiment, the ventricular tachycardia is identified based upon the tachycardia cycle length and the difference in timing between two or more electrodes. This enables a more accurate “fingerprint” to identify specific ventricular tachycardias. In general, it is optimal to first deliver overdrive pacing from the ventricular electrode with the latest activation during the ventricular tachycardia. In macro-reentrant tachycardias, this electrode is most likely to be closest to the entrance site of the reentrant tachycardia to enable identification of ventricular entrainment during overdrive pacing. If a certain overdrive pacing combination of long-short intervals is successful in terminating a specific ventricular tachycardia, another embodiment enables this combination of pacing pulses to be utilized faster in the pacing algorithm to terminate the ventricular tachycardia more quickly. In another embodiment, if overdrive pacing fails to terminate the tachycardia or does not permit monitoring and sensing of ventricular entrainment, a different pacing electrode may be utilized.

Overdrive pacing may sometimes create other reentrant tachycardias or reentry beats that complicate tachycardia termination. For example, overdrive pacing from the right ventricular apex often blocks within the right bundle branch resulting in bundle branch reentry during or after delivery of pacing pulses. Therefore, in another embodiment, overdrive pacing from an atrial electrode may be delivered while ventricular overdrive pacing is also being delivered. Measuring the conduction time from atrial pacing to his-purkinje and right ventricular electrodes when not in the tachycardia may be incorporated such that atrial overdrive pacing is performed simultaneously with ventricular pacing in order to block potential reentry beats from the exiting into the ventricular myocardium. In some circumstances, atrial pacing may be timed such that the wavefront exiting the normal conduction system is timed to facilitate tachycardia termination.

In yet another embodiment, a second electrode may not be adequately positioned on the opposite side of a reentrant tachycardia. However, by analyzing the far-field morphology of a second electrode, tachycardia entrainment can still be identified and used to guide overdrive pacing. For example, the ventricular signal from a large electrode, such as the shocking coil found in the right ventricular implantable cardioverter-defibrillator lead, measures ventricular activity distant from the electrode itself. After obtaining tachycardia entrainment, delivery of a pacing pulse with a pacing cycle length close to the tachycardia cycle length will result in more ventricular myocardium being activated by the depolarizing wavefront leaving the exit site of the reentrant circuit. The morphology of the ventricular signal can be compared between the ‘short’ or ‘accelerating’ pacing pulse with the ‘long’ or ‘priming’ pacing pulse. If these two ventricular signals are significantly different, this finding would indicate tachycardia entrainment has been obtained. Therefore, by alternating long and short pacing pulses, the morphology of the ventricular signal from the second electrode can be used to guide overdrive pacing.

For example, if the ‘short’ or ‘accelerating’ pacing pulse terminates the ventricular tachycardia, the subsequent pacing morphology of the next pacing pulse will resemble the pacing morphology of the same electrode morphology when not in a ventricular tachycardia. Furthermore, in some circumstances, the second electrode may sense ventricular depolarization occurring prior to delivery of the ‘long’ or ‘priming’ pacing pulse. Therefore, in these circumstances, overdrive pacing can be stopped on the same pacing pulse that terminates the tachycardia. In other circumstances (depending on the ventricular substrate and electrode positioning), another pacing pulse may by delivered after tachycardia termination; however, changes in the ventricular signal will indicate the tachycardia has been terminated.

Therefore, in yet another embodiment, ventricular tachycardia can be terminated by delivering overdrive pacing with at least two pacing pulses with a pacing cycle length of less then 90% of the tachycardia cycle length; followed by deliver of at least one ‘long’ pacing pulse with a pacing cycle length of greater than 90% of the tachycardia cycle length; while sensing the ventricular signals from the at least one second electrode during the delivery of overdrive pacing; in order to adjust the delivery of overdrive pacing based on the ventricular signals from the at least one second electrode.

In another embodiment, the ventricular signal from the second electrode is recorded when pacing from the stimulating electrode when not in a ventricular tachycardia and used as a reference. In another embodiment, the morphology of ventricular signals from the second electrode is compared between the pacing pulses during overdrive pacing to adjust the delivery of overdrive pacing. In another embodiment, the delivery of overdrive pacing includes alternating the at least one ‘long’ pacing pulse with a pacing cycle length greater than 90% of the tachycardia cycle length followed by at least one ‘short’ pacing pulse with a pacing cycle length less than 90% of the tachycardia cycle length.

In yet another exemplary embodiment, the systems herein may be used for overdrive pacing with at least two pacing pulses delivered from a first or pacing electrode with a pacing cycle length of less then 90% of the tachycardia cycle length delivered, followed by delivery of at least one ‘long’ pacing pulse with a pacing cycle length of greater than 90% of the tachycardia cycle length; where the ventricular signals are sensed during the delivery of the pacing pulses to adjust the delivery of overdrive pacing. When ventricular signals are sensed from the first electrode (the same electrode that is delivering overdrive pacing), the ventricular signal should include far-field morphology, or utilize a distant electrode (greater than one centimeter away from the first electrode) as an anode (as opposed to sensing in a bipolar electrode orientation).

In one embodiment, after delivery of at least one pacing pulse with a pacing cycle length less than 90% of the tachycardia cycle length, the morphology of the ventricular signal from the pacing electrode is recorded. Initial pacing pulses are delivered in efforts to entrain or terminate the ventricular tachycardia. Then, at least one pacing pulse with a pacing cycle length of greater than 90% of the tachycardia cycle length is delivered. The ventricular signal is compared between the two pacing cycle pulses using different pacing cycle lengths. If the two morphologies are significantly different, this would suggest the tachycardia remains entrained, and therefore at least one additional pacing pulse with a pacing cycle length of less than 90% of the tachycardia cycle length should be delivered. If the two morphologies are similar or identical, this would suggest either the tachycardia has been terminated or that the ventricular signal cannot be used to adjust the pacing pulses. In this scenario, the overdrive-pacing algorithm can switch to an algorithm that does not utilize the ventricular signals to adjust the pacing pulses. Alternatively, a different pacing electrode than the first electrode may be chosen to deliver the pacing pulses.

In another embodiment, the sensed ventricular signal can be analyzed during the initial pacing pulses to estimate whether the tachycardia has been entrained. In this embodiment, after delivery of at least two pacing pulses with a pacing cycle length less than 90% of the tachycardia cycle length, the morphology of the ventricular signals is analyzed to determine if tachycardia entrainment has been obtained. After evidence of tachycardia entrainment (or after delivery of enough initial pacing pulses that the tachycardia is likely entrained), then at least one pacing pulse with a pacing cycle length of greater than 90% of the tachycardia cycle length is delivered. The ventricular signal is compared between the pacing pulse with a pacing cycle length of greater than 90% of the tachycardia cycle length to the pacing pulse with a pacing cycle length of less than 90% of the tachycardia cycle length. Comparing the ventricular signals can be used to estimate tachycardia entrainment, tachycardia termination, or the inability of the ventricular signal to optimally adjust overdrive pacing.

The pattern of alternating at least one short pacing pulse (with a pacing cycle length less than 90% of the tachycardia cycle length) followed by at least one long pacing pulse (with a pacing cycle length greater than 90% of the tachycardia cycle length) can be repeated indefinitely while the sensed ventricular signals are used to identify tachycardia termination. The pacing cycle length of the ‘short’ pacing pulse can be shortened over each iteration to increase the probability of tachycardia termination. By slowly decreasing the pacing cycle lengths of the ‘short’ pacing pulse, the probability of accelerating the ventricular tachycardia or inducing ventricular fibrillation is minimized. In order to determine the initial number of pacing stimulations required to obtain tachycardia entrainment at the onset of overdrive pacing, the morphology of the ventricular signals can be analyzed to estimate the timing of tachycardia entrainment.

In another exemplary embodiment, a second or distal sensing electrode (greater than one centimeter away from the first or pacing electrode) is used to sense ventricular signals in order to adjust overdrive pacing. This distal electrode may be used as a bipolar or unipolar electrode. During the initial pacing pulses, the time delay between the pacing pulse and the sensed ventricular signal from the distal electrode is sensed and used to adjust the overdrive pacing. Once the time interval between the pacing pulse and the sensed ventricular signal is stable, this suggests 1) the tachycardia is entrained, 2) the tachycardia has been terminated, or 3) the electrode cannot determine if the tachycardia is entrained or terminated at this pacing cycle length. In one embodiment, this time interval is previously recorded when the patient is not in ventricular tachycardia and utilized to determine if this electrode can be used to identify tachycardia entrainment.

In another embodiment, delivery of a pacing pulse with a pacing cycle length greater than 90% of the tachycardia cycle length is delivered. The time interval between the pacing stimulation and the sensed ventricular signal from the second electrode can again be measured. Comparing this time interval to the time interval recorded on the previous pacing pulse (when utilizing a pacing cycle length less than 90% of the tachycardia cycle length), it can be used to determine one of the following: 1) the tachycardia is entrained, 2) the tachycardia has been terminated, or 3) the electrode cannot determine if the tachycardia is entrained or terminated. If the tachycardia is identified as being entrained, at least one additional pacing pulse with a pacing cycle length of less than 90% of the tachycardia cycle length can be delivered, again while monitoring the time interval between the pacing pulse and the sensed ventricular signal.

In some circumstances, noise artifact (or blanking periods) limit the ability to sense ventricular signals from the second electrode. However, in most circumstances, the time interval between the pacing electrode and the sensing electrode when not in tachycardia is sufficiently long enough to permit sensing the ventricular signal. Therefore, even if noise or blanking prevents identification of the ventricular signal, the lack of a ventricular signal in the time interval that would be expected when not in a tachycardia can be monitored. Therefore, if no ventricular signal is sensed within this expected window, the lack of a signal suggests tachycardia entrainment is still present. Therefore, sensing from a second electrode can still be used to guide overdrive pacing even when the majority of sensed ventricular signals fall within a required blanking period. The use of a pacing pulse with a pacing cycle length greater than 90% of the tachycardia facilitates ventricular sensing. Furthermore, by recording the conduction time from the pacing electrode to the sensing electrode when not in tachycardia, this time interval can be used as a benchmark to determine tachycardia entrainment during overdrive pacing. If a ventricular signal is sensed in this window, overdrive pacing can be stopped.

In some circumstances, the second electrode cannot be used to adjust overdrive pacing. In this circumstance, the return interval from the pacing electrode (the post-pacing interval) can be used to determine the entrainment time required to entrain the tachycardia. The entrainment time can be used to estimate the time delay within the reentrant circuit. In yet another embodiment, if during the initial overdrive pacing the sensed ventricular signal continues to change, overdrive pacing can continue pacing at the original overdrive pacing cycle length until the interval between the pacing stimulation and the sensed ventricular signal is stable. In yet another embodiment, comparing the changes in the sensed ventricular signal during overdrive pacing can be used to estimate the time delay that occurs within the reentrant circuit. This estimated time delay (or a percentage of this value) can be added to the tachycardia cycle length (or a percentage of the tachycardia cycle length) to determine the pacing cycle length of the ‘long’ or ‘priming’ pacing pulse. Therefore, during the alternating ‘long’ and ‘short’ pacing pulses, the ‘short’ pacing pulse is gradually shortened.

The ‘short’ pacing pulses often cause conduction delay within the reentrant circuit prior to tachycardia termination. As the ‘short’ pacing pulses continue to prolong the sensed time interval between the stimulation and the sensed ventricular signal, the time delay continues to prolong. This longer time delay can be added to the ‘long’ pacing pulse to deliver even longer ‘long’ pacing pulses. The longer this time the slower the rate of repolarization on the next pacing stimulation, since the rate of depolarization largely determines the rate of repolarization. Therefore, the ‘long’ pacing intervals slowly get longer while the ‘short’ pacing intervals slowly get shorter. Both of these changes increase the probability of tachycardia termination.

In accordance with another embodiment, a system is provided for delivering therapy to terminate a tachycardia event in a heart of a patient that includes a pacing device configured to be implanted in the patient's body and comprising a processor; a plurality of electrodes coupled to the processor and sized for implantation within the patient's body; wherein the processor is configured to: measure a time interval from stimulation from a first electrode to a sensed ventricular signal from at least one second electrode when the patient is not in ventricular tachycardia; detect a ventricular tachycardia having a tachycardial cycle length (TCL); deliver overdrive pacing from one or more electrodes in response to the detection; sense ventricular signals from the at least one second electrode during the delivery of overdrive pacing; and adjust the delivery of overdrive pacing based on the sensed ventricular signals from the at least one second electrode. In addition or alternatively, the processor and/or system may be configured to treat tachycardia using any of the embodiments described above.

Other aspects and features of the present invention will become apparent from consideration of the following description taken in conjunction with the accompanying drawings.

### BRIEF DESCRIPTION OF THE DRAWINGS

In general, examples disclosed herein are directed to treating tachyarrhythmia, such as ventricular tachycardia, by employing at least one electrode to deliver electrical stimulation to a patient's heart in a manner designed to terminate a tachyarrhythmia episode.

FIG. 1 shows a schematic diagram of an exemplary pacemaker/implantable cardio-defibrillator (ICD) with leads introduced into regions of a heart.

FIG. 2 is an exemplary embodiment of a functional block diagram of circuitry that may be provided in the implantable pacemaker/cardioverter/defibrillator of FIG. 1.

FIG. 3 is a simplified diagram of a reentrant tachycardia circuit within a heart and a single pacing electrode to demonstrate the different timing intervals.

FIG. 4 is a simplified diagram of a reentrant tachycardia circuit within a heart and two pacing electrodes for sensing and/or delivering electrical stimulations according to an exemplary embodiment.

FIG. 5 is a simplified diagram of a reentrant tachycardia circuit within a heart and a single pacing electrode, which has entrained the tachycardia, using different pacing outputs.

FIG. 6 is a graphical representation of anti-tachycardia pacing (ATP) according to an exemplary embodiment.

### DETAILED DESCRIPTION OF THE EXEMPLARY EMBODIMENTS

Overdrive pacing is typically performed at a rate faster (e.g., pulse rate frequency) than the native tachycardia; therefore the paced cycle length of the pacing (“PCL”) is usually shorter than the tachycardia cycle length (“TCL”), i.e., PCL<TCL. Each paced stimulation ‘gains’ on the native tachycardia by the amount of pacing prematurity, or the difference between the tachycardia cycle length and overdrive pacing cycle length (TCL-PCL). Overdrive pacing continues to ‘gain’ on the native tachycardia until the paced wave front reaches and then accelerates the native tachycardia. Once overdrive pacing reaches the arrhythmic circuit, additional pacing stimulations continuously reset the native tachycardia to the PCL (entrainment).

After obtaining entrainment, the pacing electrode can deliver a single or multiple stimulations at the original tachycardia cycle length. If this occurs, the pacing wavefront will reach the reentrant circuit (i.e., the region within the heart tissue causing the tachycardia) at approximately the same time as the native wavefront traveling in the reentrant circuit. Importantly, when pacing close to the tachycardia cycle length, the tachycardia cycle length determines the repolarization rates within the tachycardia circuit. Therefore, an ATP attempt can deliver stimulations at a slower cycle length (such as the original tachycardia cycle length) in order to delay the rate of repolarization.

For example, using an implanted system, such as those described elsewhere herein, an ATP attempt may deliver a first set of eight stimulations at PCL1 (a paced cycle length shorter than the tachycardial cycle length TCL), e.g., via one or more pacing electrodes of the system. In response to the stimulations, far-field morphology and a distal electrode may identify the tachycardia has been entrained. Once entrained, the ATP algorithm can then deliver a second set including one or several pacing stimulations at the original tachycardia cycle length (e.g., CL2 of the second set approximately equal to or longer than TCL). This slower rate will delay the rate of tissue repolarization; however, pacing at or near the native TCL maintains access that the pacing electrode has with the reentrant tachycardia. The ATP algorithm can then deliver a third set including a single or several stimulations at a shorter cycle length (CL3 of the third set less than TCL) that rapidly and completely accelerate the tachycardia. Far-field morphology and the distal electrode can identify if the tachycardia has been terminated.

If the tachycardia has not been terminated, the device can deliver a fourth set including a single or several stimulations at the tachycardia cycle length (e.g., CL4 of the fourth set approximately equal to or longer than TCL), followed by a fifth set including a single or several stimulations at an even shorter cycle length than the previous attempt (i.e., CL5 of the fifth set being less than CL3). This pattern can be repeated at shorter and shorter cycle lengths (i.e., stimulation sets at TCL followed by stimulations sets “n” where CL(n)<CL(n−1)) until the tachycardia is terminated. In this manner, only a single ATP attempt is required to terminate the tachycardia and also improves the ability to terminate the tachycardia at any given paced cycle length.

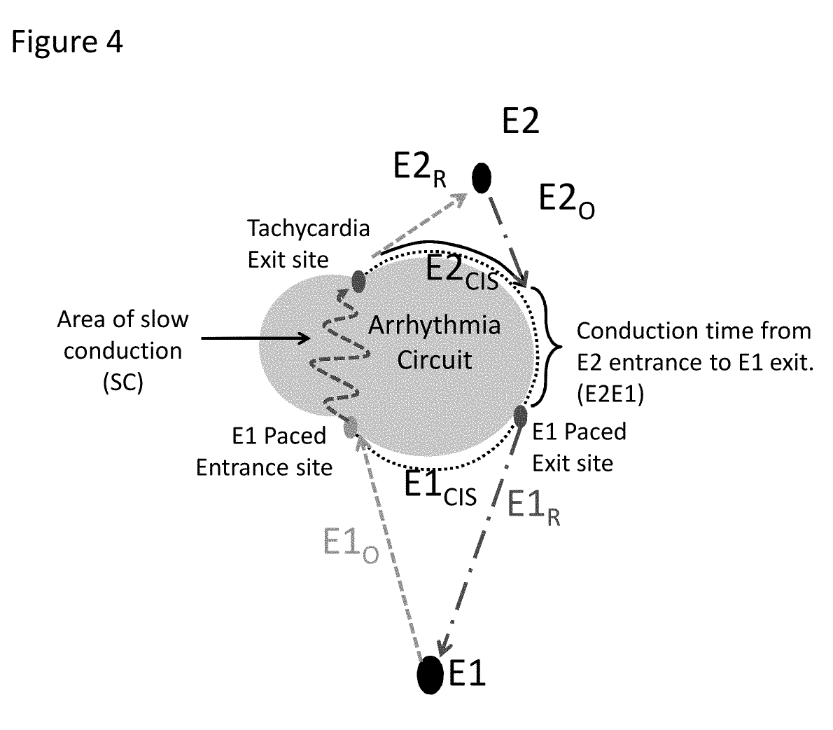

Additionally, pacing may advance the tachycardia through critical aspects of the reentrant circuit. However, when the diastolic potential is shortened, the conduction velocity may slow. Therefore, a single accelerating pacing stimulation may advance proximal portions of the reentrant circuit; however, conduction slowing protects more distal locations. Therefore, continuous pacing may be required to propagate the pacing prematurity to the most sensitive/critical aspects of the reentrant circuit. Furthermore, even after the pacing prematurity has propagated through critical aspects of the reentrant circuit, the tachycardia may not terminate. However, by measuring the changes in timing interval during the ATP attempt, or by measuring differences in timing intervals during ATP attempts from two or more pacing electrodes (see, description associated with FIG. 4 elsewhere herein for more details), a priming pulse may be delivered, whereby the conduction delay can be estimated and added to the TCL in order to prolong the recovery time of critical aspects of the reentrant circuit. The prolonged diastolic period will delay repolarization and can be used to facilitate tachycardia termination.

In another embodiment, the interval between stimulation to far-field morphology is measured to estimate the distance the pacing electrode is from critical aspects of the reentrant circuit. The time required for the last paced beat to create a wavefront, enter the arrhythmic circuit, propagate around a portion of the arrhythmic circuit, and then exit to the same electrode or catheter is termed the post-pacing interval (PPI). That is, the PPI-TCL is often measured after entrainment maneuvers to estimate the distance between the pacing electrode and the reentrant circuit.

However, by measuring the relative distance from a pacing electrode to the critical aspects of the reentrant circuit, the electrode closest to the critical isthmus can be determined. As will be further discussed in reference to FIG. 4, an electrode may have a short PPI-TCL; however, the electrode may be relatively far from the critical isthmus maintaining the tachycardia. Therefore, the stimulation to QRS during entrainment or other similar analyses can be performed to estimate the electrode that is closest in time to the critical isthmus.

A distal sensing electrode and far-field morphology may or may not identify entrainment and/or tachycardia termination. These abilities are dependent on a number of details specific to the tachycardia including spatial relationships between electrodes and the native tachycardia and conduction properties within the myocardium. Fortunately, bi-ventricular pacing systems usually incorporate two electrodes, which are distant from each other. Having two electrodes distant to each other across the myocardium increases the probability that these electrodes will be able to determine the timing of tachycardia entrainment, tachycardia termination, and the amount of time delay within the reentrant tachycardia. In addition, left ventricular pacing leads frequently contain four different pacing electrodes. Therefore, the probability that one of the electrodes can be used to determine these timing intervals is increased.

One exemplary embodiment describes far-field morphologies, tachycardia rates, and timing differences between electrodes to identify various reentrant tachycardias a patient may have. By accurately identifying the varying reentrant tachycardias, prior ATP failures and attempts can used to optimize subsequent ATP attempts.

In yet another embodiment, the pacing output is adjusted to optimize tachycardia termination. For example, a higher pacing output, i.e., electrical power output, captures more local myocardium around the electrode than pacing at a lower pacing output. Therefore, the power output can be adjusted during overdrive pacing to advance the tachycardia without changing the pacing cycle length. By capturing more myocardium during pacing, the conduction time from the pacing electrode to the reentrant circuit is decreased. The higher pacing output may not be necessary until there is evidence (or increased probability) that the higher pacing output will reach the reentrant circuit. Therefore, by changing the pacing output during the delivery of ATP, battery life can be maintained. Furthermore, the power output may be significantly increased beyond what is normally delivered during ATP. For example, power output that may be felt by the patient, however, significantly lower than output utilized during defibrillation may be employed. This higher output power may reset more myocardium, advance to critical aspects of the tachycardia more quickly, and ultimately be more effective in terminating the tachycardia. In another embodiment, the power output is decreased during overdrive pacing. The power output is decreased during overdrive pacing in order to have better sensing of the ventricular signal, since higher power output can saturate the sensing capabilities of the system and distort the ventricular signal. By decreasing the power output, the sensed ventricular signal can be more useful.

In yet another embodiment, a second sensing electrode (or a plurality of such sensing electrodes) located greater than one centimeter (1 cm) away (distal) from the pacing electrode (used to deliver the overdrive pacing) can be used to monitor the ATP attempt. Ideally, this sensing electrode is located on the other side of the reentrant tachycardia. For example, the pacing electrode may be the right ventricular pacing lead while the sensing electrode may be the left ventricular pacing lead of a biventricular pacing system. Depending on the direction of the ventricular tachycardia (the pacing electrode is more effective if positioned near the entrance site of the tachycardia), the left ventricular lead may be the pacing electrode and the right ventricular lead may be the sensing electrode.

The time intervals sensed during the delivery of overdrive pacing can be used to determine if the sensing electrodes (located greater than centimeter away) are advantageously positioned to confirm tachycardia entrainment and/or tachycardia termination and therefore be used to guide the overdrive pacing. Time changes in the tachycardia cycle length during pacing are used to adjust the next stimulated pacing cycle length. Once entrainment has been verified and the conduction delay is stable (without tachycardia termination), the ATP algorithm can deliver one or more stimulations close to the baseline tachycardia cycle length plus some percentage of the measured time delay within the circuit (or a function related to one or both measurements), followed by one or more stimulations at a shorter pacing cycle length, e.g., as described further elsewhere herein. The long pacing cycle lengths serve to ‘prime’ the reentrant tachycardia to prolong repolarization times in order to facilitate tachycardia termination.

In yet another embodiment, fluctuations in conduction intervals may be used to understand the phase of the tachycardia. More specifically, harmonic variations can be measured and utilized to guide the timing and output of anti-tachycardia pacing stimulations.

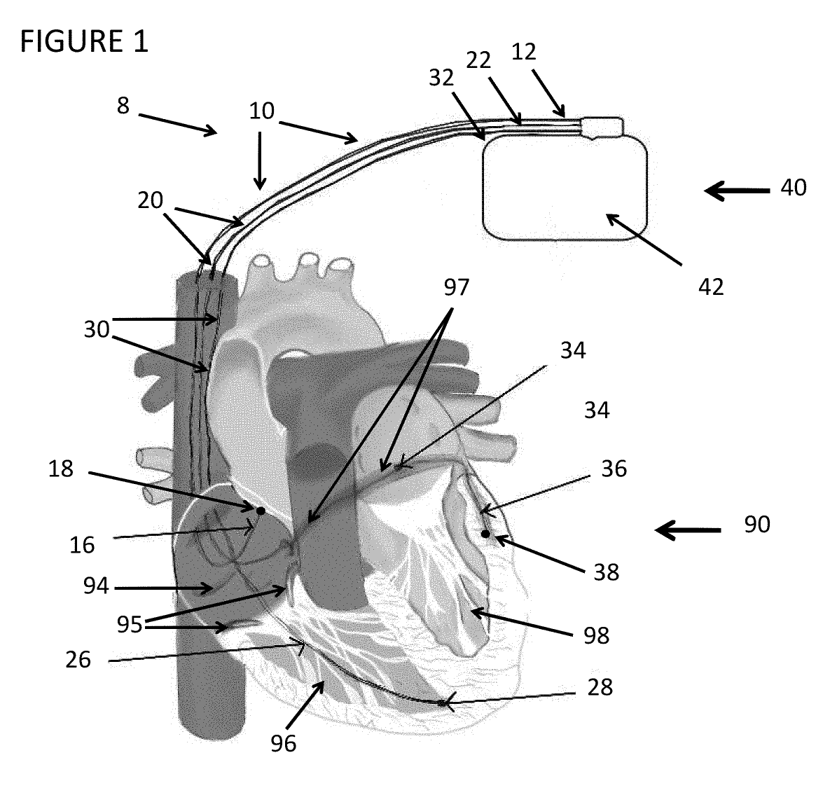

FIG. 1 shows an exemplary embodiment a pacemaker/implantable cardio-defibrillator (ICD) 8 with leads introduced, implanted, or otherwise positioned within chambers or vessels of a heart 90. In the embodiment shown in FIG. 1, the ICD 10 may include a controller 40 sized to be implanted within the patient's the body, e.g., subcutaneously adjacent the heart 90. The controller 40 may include a housing 42 connected to a plurality of leads 10, 20, 30 that are sized such that at least a portion of the leads 10, 20, 30 may be implanted into the patient's heart 90.

The first lead 10 may include a proximal end 12 coupled to the housing 42 and a second end 16 sized for introduction into the patient's heart 90, e.g., into the right atrium 92. The distal end 16 of the first lead 10 may carry one or more sensors and/or electrodes 18 configured to sense electrical activity (depolarizations) and/or pace the right atrium 92, as programmed. In addition, the distal end 16 of the first lead 10 may include one or more features, e.g., a screw tip or other anchor (not shown) on the distal end 16 for securing the distal end 16 relative to the right atrium 92 or left atrium (not shown). In an exemplary embodiment, one or more wires or other conductors (not shown) may extend from the distal end 16 to the proximal end 12 to communicate signals from a sensing electrode to the controller 40.

Similarly, the second lead 20 may include a proximal end 22 coupled to the housing 42 and a second or distal end 26 sized for introduction into the patient's heart 90, e.g., into the right atrium 92, through the tricuspid valve 95 and into the right ventricle 96. The distal end 26 which may carry one or more electrodes, e.g., electrode 28 designed to sense electrical activity or deliver electrical energy to stimulate the right ventricle 96. The second lead 20 may also carry a shocking coil (not shown). In order to sense an electrical signal, the controller 40 requires a cathode and an anode, i.e., a positive and negative ‘end’ to measure the electrical activity.

In one embodiment, a single sensing electrode 28 may be provided on the distal end 26, e.g., to provide unipolar signals to the controller 40. In this embodiment, the system 8 may include another component as an anode, e.g., the housing 42 of the generator 40. In another embodiment, multiple sensing electrodes (not shown) may be provided on the distal end 26, e.g., to provide bipolar electrodes, e.g., two or more electrodes that are in close proximity to each other (typically within a few millimeters of each other) and the controller 40 obtains signals from one or more pairs of the electrodes. Alternatively, multiple electrodes may be provided that are configured as unipolar electrodes.

The larger the distance between the anode and cathode, the greater the distance electrical signals are recorded. Therefore, bipolar electrodes sense electrical activity in close proximity to the two electrodes, while unipolar electrodes measure electrical activity from farther away, or far-field signals. In addition, the ventricular signals from the shocking coil often record ventricular signals that occur from farther away.

Similar to the first lead 10, the second lead 20 may include one or more features, e.g., a screw tip or other anchor (not shown), on the distal tip to secure the distal end 26 within the patient's heart 90, e.g., the wall of the heart 90 within the right ventricle 96, similar to pacing leads. Alternatively, the first and second leads 10, 20 may be provided on a single device with a branch distal end (not shown), similar to systems shown in U.S. Publication No. 2016/0199554, the entire disclosure of which is expressly incorporated by reference herein.

Additionally, the system 8 may include a third lead 30 including a proximal end 32 coupled to the housing 42 and a second or distal end 36 sized for introduction into the patient's heart 90, e.g., into the right atrium 92, through the coronary sinus 97 or other vein of the heart 90, e.g., into a coronary vein. The third lead 30 may include one or more sensors or electrodes, e.g., configured to sense or deliver electrical activity. For example, as shown, a first sensor or electrode 34 may be provided at an intermediate location of the lead 30, e.g., such that the first electrode 34 may be positioned adjacent the left atrium, to sense and/or deliver electrical activity from/to either atrial or ventricular tissue. In addition, the third lead 30 may include a second sensor or electrode 38 carried on or adjacent a distal tip of the distal end 36 to sense and deliver electrical activity from/to a different location in the heart 90, e.g., adjacent the left ventricle 98. Similar to the first lead 10 and second lead 20, the third lead 30 may include one or more features, e.g., a screw tip or other anchor (not shown), on the distal tip to secure the distal end 36 within the patient's heart 90, e.g., within a distal vein of the coronary sinus 97, similar to typically used pacing leads.

In an exemplary embodiment, the third lead 30 may be coupled to the controller 40 for sensing and/or pacing electrical activity occurring in the left ventricle 98. In addition, if there is a tachycardia occurring within the right ventricle 96 or the left ventricle 98, the sensors or electrodes 28, 34, or 38 may sense and/or deliver electrical activity in order to terminate the tachycardia. In addition, if there is a tachycardia occurring within the right atrium 94 or left atrium (not shown), the sensors or electrodes 18 or 34 may sense and/or deliver electrical activity to terminate the tachycardia. The electrodes 18, 28, 34, or 38 are used to illustrate the delivery of anti-tachycardia pacing (ATP) therapy throughout this description, although any number of electrodes are capable of sensing and delivering electrical energy to the heart 90. The ATP therapy may be used to treat ventricular as well as atrial tachyarrhythmias.

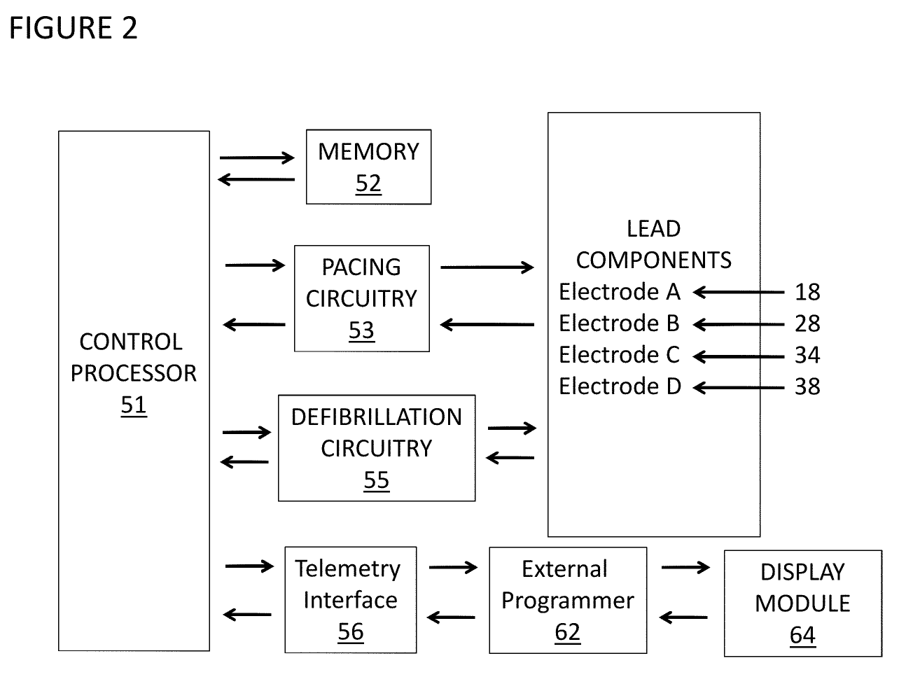

FIG. 2 shows a simplified functional block diagram of an exemplary embodiment of components that may be located within the housing 42 and/or otherwise connected to the controller 40. As shown, the components include a control processor 52 which receives input information from various components, e.g., sensors or electrodes of the leads 10, 20, 30, in order to determine the pacing algorithm in order to treat the patient. The control processor 52 is connected to pacing circuitry 53, defibrillation circuitry 55, memory 52, and a telemetry interface 55. The pacing circuitry 53 and the defibrillation circuitry 55 connect to the first lead 10, second lead 20, and third lead 30 via one or more wires or conductors (not shown); and ultimately connects to the electrodes, for example, 18, 28, 34, and 38.

These connections allow for multiple capacities to sense electrical activity (such as myocardial depolarizations), deliver pacing stimulations, and/or deliver defibrillation or cardioversion shocks. For example, based on the input(s) received from the electrodes 18, 28, 34, and 38 through the pacing circuitry 53, the control processor 51 performs calculations to determine the proper course of action, which may include providing ATP therapy to one or more electrodes, providing defibrillation or cardioversion shocks to one or more electrodes through the defibrillation circuitry 55, or no therapy at all, e.g., using any of the embodiments described elsewhere herein.

Optionally, the control processor 51 is connected to a telemetry interface 56, e.g., within the housing 42. The telemetry interface 56 can wirelessly send and/or receive data to and/or from an external programmer 62, which may be coupled to a display module 64 in order to facilitate communication between the control processor 51 and other aspects of the system external to the patient.

Upon implantation, the control processor 51 may continuously or intermittently sense electrical activity from one or more electrodes, e.g., while delivering pacing stimulations to other electrodes when treatment is needed. The control processor 51 may store selected data to memory 52, and retrieve stored data as necessary. For example, the control processor 51 may identify key aspects of a tachyarrhythmia in order to effectively differentiate the tachycardia and other key aspects of the tachyarrhythmia in order to optimize the best therapy in order to terminate the tachyarrhythmia.

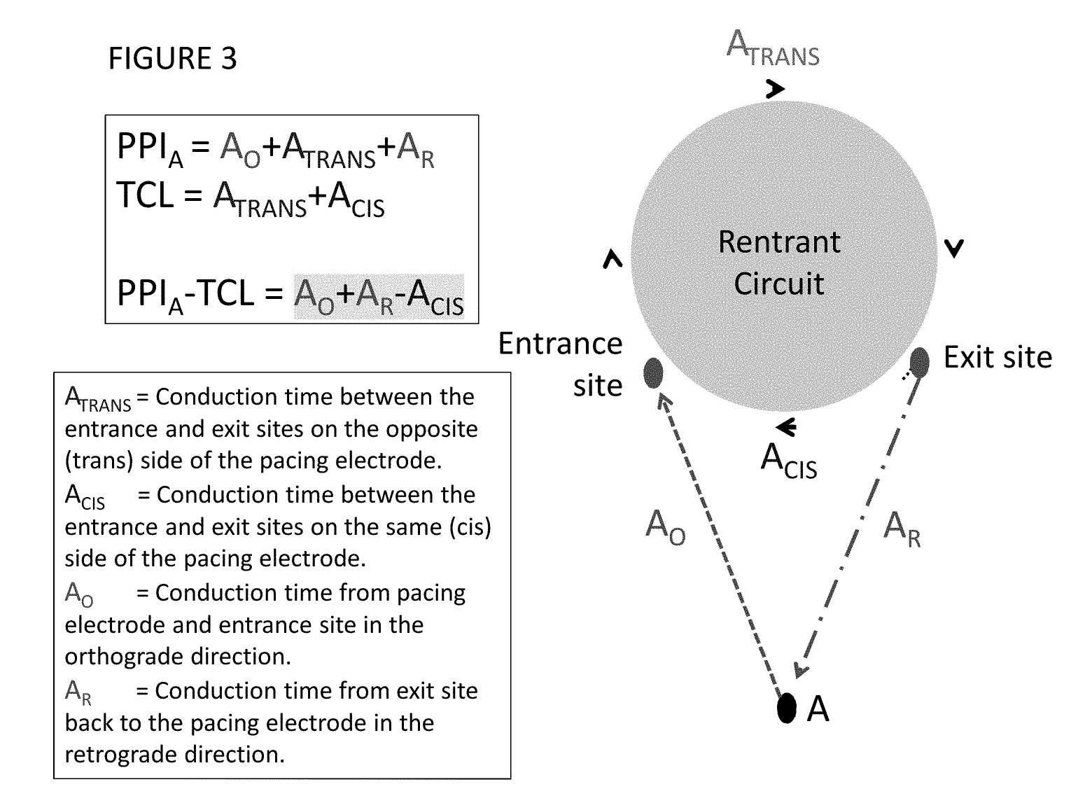

Turning to FIG. 3, a simplified diagram of a reentrant tachycardia circuit is shown within a region of a heart with a single pacing electrode to demonstrate the different timing intervals. Once overdrive pacing resets or entrains the tachycardia, the post-pacing interval (PPI) at the cessation of pacing measured from the last stimulus (S) to the next depolarization represents the conduction time from the pacing site to the reentrant circuit, through the circuit, and then back to the pacing site (e.g., via one of the electrodes shown in the system 8 of FIG. 1). Importantly, the paced wavefront may enter (‘the entrance site’) and leave (‘the exit site’) the reentrant circuit at different locations.

In some cases, such as AVRT, the distance between the entrance and exit sites may be significant. In these cases, the post-pacing interval will not include the time to travel the entire reentrant circuit. Instead, the PPI will only include the part of the TCL between the entrance and exit sites on the opposite side of the pacing catheter (Time A_{TRANS}). Note that the tachycardia cycle length (TCL) is equal to the time to travel from the entrance site to the exit site opposite the pacing catheter (Time A_{TRANS}) plus the time to travel from the exit site to the entrance site on the same side as the pacing location (Time A_{CIS}). When calculating the PPI-TCL, note the Time A_{TRANS} cancels, as seen in Equations 1-3 below.

PPI_{A}=A_{O}+A_{TRANS}+A_{R} (1)

TCL=A_{TRANS}+A_{CIS} (2)

PPI_{A}−TCL=A_{O}+A_{R}−A_{CIS} (3)

The time to travel from the entrance site to the exit site (Time A_{TRANS}) is assumed to be equal during tachycardia as well as overdrive pacing. However, the conduction velocity through the reentry circuit may change during overdrive pacing. If the conduction velocity slows, the PPI prolongs. Therefore, the PPI-TCL might be understood more precisely using equation 4 below.

PPI_{A}−TCL=A_{O}+A_{R}−A_{CIS}+(A_{TRANS}^{@PCL}−A_{TRANS}^{@TCL}) (4)

As demonstrated by equation 4, unadjusted measurement of the PPI is influenced by changes in conduction velocity as a result of different pacing rates.

Turning to FIG. 4, a simplified diagram is shown of a reentrant tachycardia circuit within a heart and an exemplary system (e.g., the system 8 of FIG. 1) including two pacing electrodes E1, E2 for sensing and/or delivering electrical stimulations. Since the PPI-TCL is determined using measurements at both the TCL and the pacing rate, one method to determine the total amount of decremental conduction is by measuring various time intervals using multiple electrodes. As demonstrated in FIG. 4, the time intervals during and after entrainment can measure key timing intervals as demonstrated below:

StimE1→E2=E1_{O}+SC+E2_{R} (5)

StimE2→E1=E2_{O}+E2E1+E1_{R} (6)

PPI_{E1}=E1_{O}+SC+E2_{CIS}+E2E1+E1_{R} (7)

PPI_{E2}=E2_{O}+E2E1+E1_{CIS}+SC+E2_{R} (8)

PPI_{E1}+PPI_{E2}−[StimE1−E2]−[StimE2−E2]=SC+E2_{CIS}+E2E1+E1_{CIS} (9)

Note that equation 9 is the total conduction time required to travel around the reentrant circuit when the pacing cycle length is at the overdrive pacing cycle length (PCL). Therefore, the difference between the measured value of equation 9 and the TCL may be used to estimate the total amount of conduction delay within the reentrant circuit. In another embodiment, assumptions regarding the location of conduction delay can be made to estimate the total amount of decremental conduction in each component. FIG. 4 also demonstrates how the key time components that determine the PPI-TCL can also be determined using time components from two entrainment maneuvers. Importantly, all time measurements using this method are measured at the same overdrive pacing cycle length.

In FIG. 4, note there is an area of slow conduction. When monitoring an ATP attempt, the conduction through the area of slow conduction should be monitored. Therefore, if ATP is delivered from electrode E2, monitoring the conduction at electrode E1 will not be as helpful as performing ATP from electrode E1 and monitoring from electrode E2 since orthograde conduction is most likely to occur at the location of conduction delay—which is typically located at the location of the critical isthmus. When performing ATP from electrode E1, the timing intervals at electrode E2 can be used to estimate, among other important parameters, the timing of tachycardia entrainment, conduction delay within the critical isthmus, conduction block within the critical isthmus, and tachycardia acceleration. Note that when the patient is not in tachycardia, there are shorter pathways to travel from electrode E1 to E2. However, during tachycardia, there will be wavefront collision preventing a shorter conduction interval from electrode E1 to E2, except through the critical isthmus. Also note that given three-dimensional characteristics of ventricular myocardium, there may be additional pathways that need may need to be incorporated into the pacing algorithm. Therefore, various time intervals and far-field morphology analysis are important to determining the relative proximity a pacing electrode is from the critical isthmus in the orthograde direction. Interestingly, performing ATP close to an exit site may actually protect the critical isthmus from termination (retrograde infiltration will increase the amount of repolarization time available for the tissue involved).

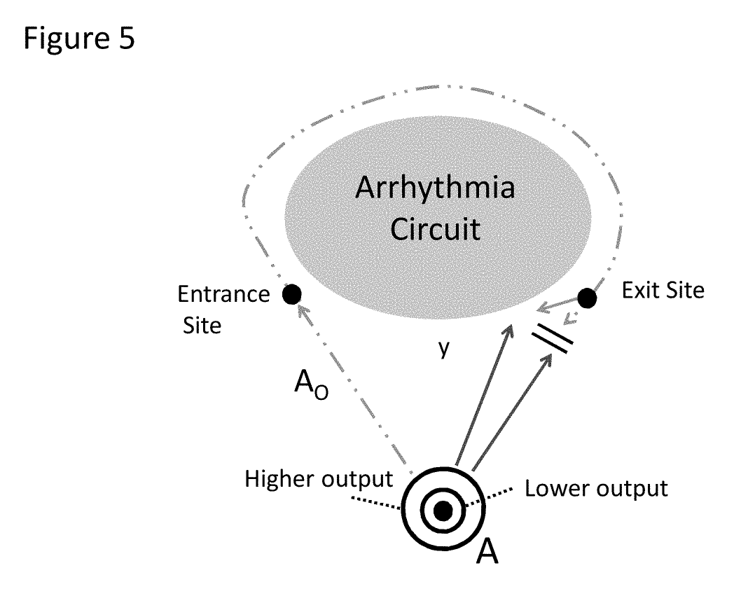

Turning to FIG. 5, a simplified diagram is shown of a reentrant tachycardia circuit within a heart and a system including a single pacing electrode A, which has entrained the tachycardia using different pacing outputs. As described elsewhere herein, ATP typically terminates a reentrant tachycardia by delivering a wavefront to the critical isthmus before the tissue has repolarized. Therefore, methods and systems are described herein to shorter the time interval from the pacing electrode to the critical isthmus of the ventricular tachycardia. In addition, repolarization and restitution properties of myocardium are taken into consideration to facilitate tachycardia termination.

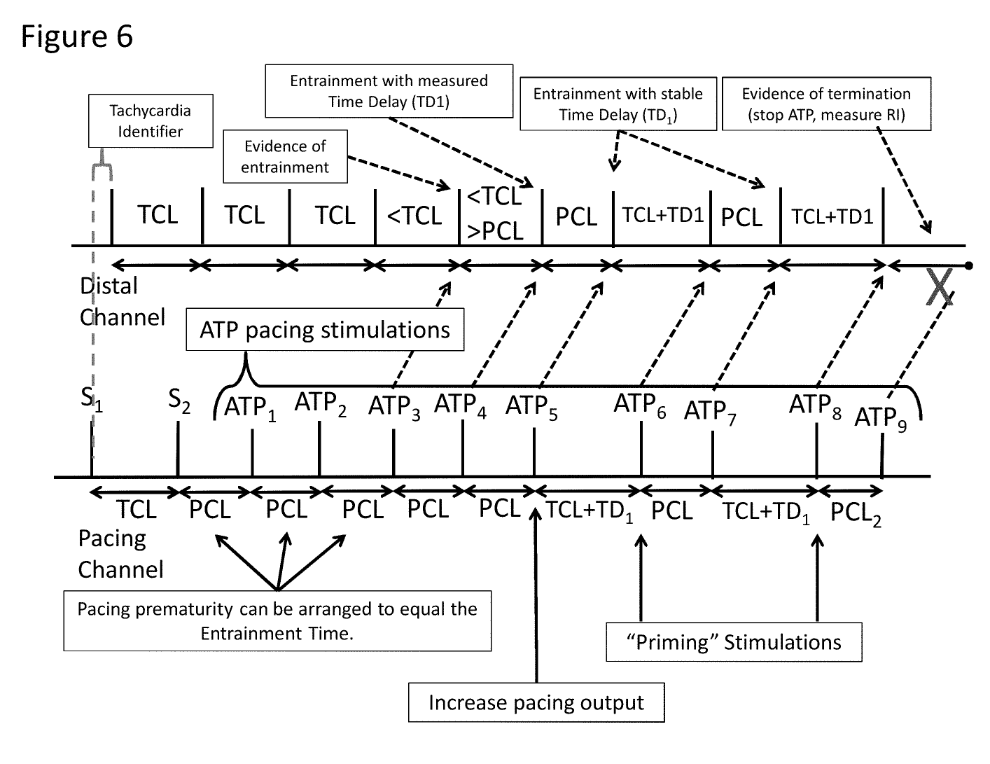

As demonstrated in FIG. 5, when stimulating electrode A, a wavefront is created that expands outwardly. However, if higher power is used to stimulate the myocardium, a larger area of myocardium is captured. Therefore, the conduction time from the pacing electrode to the reentrant circuit is slightly affected by the pacing output power. By increasing the output, the conduction interval is shortened. In addition, more tissue around the electrode is captured. During the initial pacing stimulations, higher power for local capture may not be required. Therefore, depending on the step in the ATP algorithm (see FIG. 6 and description), the output may be altered in order to facilitate tachycardia termination.

In one embodiment, after obtaining entrainment, a higher output power is utilized to advance the paced wavefront to the most vulnerable aspects of the reentrant circuit. In other embodiments, initial ATP pulses are delivered in order to locate depolarized tissue far from the pacing electrode. Subsequently, defibrillation can be performed which has a greater probability of terminating the tachycardia. This is because successful defibrillation requires most, if not all, of the myocardium to be captured. The myocardium that may not be depolarized by defibrillation can be synched such that this tissue is depolarized as the time of defibrillation. This method may also be performed such that lower defibrillation power is required to terminate the tachycardia. Lower power may preserve battery life and be less traumatic for the patient.

In yet another embodiment, the output power that is sensed by the patient or better tolerated by the patient is recorded. This lower power is delivered at the end of or during ATP pacing in efforts to terminate the tachycardia. Analysis of the QRS to local depolarization, as well as entrainment stimulation to local QRS sensation can be recorded in order to estimate the location the reentrant circuit is from the pacing location. By estimating the distance the pacing electrode is from various aspects of the reentrant circuit (local entrance site, local exit site, critical isthmus exit site, and critical isthmus entrance site, etc.), the amount of output power delivery can be estimated in order to successfully terminate the tachycardia while causing minimal if any patient symptoms.

In another embodiment, by estimating the location of wavefront depolarization, lower defibrillation power may be used.

Turning to FIG. 6, a graphical representation is shown of anti-tachycardia pacing (ATP) according to an exemplary embodiment. Depolarizations or signals sensed by the pacing channel electrode are labeled S1 and S2. The distal channel electrode has a line when it senses depolarizations or signals. ATP pulses are labeled with ATP1 being the first pacing stimulation and ATP9 is the last pacing stimulation. The time interval between the first and second sensed signals is the tachycardia cycle length (TCL). The TCL, the timing differences between electrodes, and the far-field morphology (not shown) can be used to identify the tachycardia (“Tachycardia Identifiers”). The sensed signals can alert the Control Processor of the tachycardia and determines the ATP pacing strategy. The device begins delivering ATP pulses.

If the Time to Entrainment had previously been recorded for this tachycardia, the device would arrange the initial pacing prematurity such that the sum prematurity is equal to the Time to Entrainment. The following pacing stimulation would then completely accelerate the arrhythmic circuit by the pacing prematurity of that paced stimulation (TCL-PCL).

In FIG. 6, the third pacing stimulation brings in the sensed signal on the distal channel. Assuming this time is shorter than the previously recorded conduction time between these electrodes, this would suggest the tachycardia has been entrained. The pacing prematurities can be summed to estimate the Time to Entrainment; alternatively the difference in time from the Stim to sensed signal on the distal channel between the first entrained stimulation and the baseline tachycardia can be used to estimate the Time to Entrainment. In the graphical representation, note the fourth pacing stimulation should be completely entrained, however, there is a prolongation in the stimulation to sensed time on the distal channel. This prolongation can be attributed to conduction delay within the myocardium and can be used to estimate the Time Delay (TD1). The fifth ATP pacing stimulation notes a stable Time Delay. Therefore, in one embodiment, the device delivers a “Priming” stimulation, by delivering a stimulation with the interval of the TCL plus the TD1.

In other embodiments, this “priming” stimulation can deliver one or more pacing stimulations at or near the TCL. The device then delivers a pacing stimulation with a shorter interval. In this example, the tachycardia does not terminate (there is a sensed signal from ATP 7) and there was no measured conduction delay (the interval equals the PCL). Had the device measured a conduction delay, the device could have continued delivering ATP therapy at the same PCL. In the current embodiment, the device delivers a second “priming” stimulation, followed by a PCL at a shorter cycle length (PCL2). In this example, the sensed signal on the distal channel does not sense a signal (the amount of delay deemed significant can be programmed) and therefore can identify the tachycardia as terminated and pacing is stopped in order to assess the rhythm.

In another embodiment, output power is increased as an alternative, or in addition to, shortening the pacing stimulation interval. The higher power facilitates faster conduction time to critical aspects of the reentrant circuit. The higher power may not need to be utilized until the pacing stimulation has an increased probability of tachycardia termination. In yet another embodiment, a constant output may be delivered in order to slow conduction velocity or delay repolarization of the tissue in close proximity of the electrode.

Sometimes, the Time Delay measured occurs from conduction changes or circuit changes at locations other than the arrhythmic circuit, for example, near the pacing electrode. In this scenario, delivering priming stimulations at the TCL plus the TD1 will result in loss of entrainment to the arrhythmic circuit. The time interval between the stimulation and the sensed signal on the distal channel can be used to identify and the ATP pacing algorithm can be adjusted to correct for this.

In FIG. 6, note the sensed signals sometimes occur simultaneously with the ATP stimulations. When this occurs, crosstalk can prevent this signal from being measured. Therefore, as previously discussed, sensing from more than one sensing channels can help monitor the tachyarrhythmia during ATP therapy. Therefore, multiple sensing electrodes can be used in combination (and in conjunction with far-field morphology analyses) to assess for entrainment, conduction delay, and changes in the tachyarrhythmia (such as termination or acceleration). Therefore, in one embodiment, catheters with two or more electrodes may be provided in order to sense cardiac depolarizations from more than one location in order to avoid sensing issues (blanking) which may occur when sensing from one location.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A method for terminating a ventricular tachycardia, comprising:
measuring a time interval from stimulation from a first electrode to a sensed ventricular signal from at least one second electrode when the patient is not in ventricular tachycardia;
detecting a ventricular tachycardia having a tachycardial cycle length (TCL);
delivering overdrive pacing from one or more electrodes in response to the detection;
sensing ventricular signals from the at least one second electrode during the delivery of overdrive pacing; and
adjusting the delivery of overdrive pacing based on the sensed ventricular signals from the at least one second electrode.

2. The method of claim 1, wherein adjusting the delivery of overdrive pacing includes both adjusting timing of pacing pulses of the overdrive pacing and power output of the overdrive pacing.

3. The method of claim 2, wherein adjusting the delivery of overdrive pacing comprises delivering alternating at least one ‘long’ pacing pulse with a pacing cycle length (PCLlong) greater than 90% of the tachycardia cycle length (TCL) of the ventricular tachycardia followed by at least one ‘short’ pacing pulse with a pacing cycle length (PCLshort) less than 90% of the tachycardia cycle length.

4. The method of claim 3, further comprising repeating delivering of a ‘long’ pacing pulse followed by a ‘short’ pacing pulse one or more times, wherein each successive ‘short’ pacing pulse has a shorter pacing cycle length (PCLshort) than a previous ‘short’ pacing pulse until there is evidence of either tachycardia termination or tachycardia acceleration of the ventricular tachycardia.

5. The method of claim 3, further comprising:
estimating conduction delay within a reentrant circuit causing the ventricular tachycardia during the overdrive pacing based on one or more sensed ventricular signals from the at least one second electrode; and
using the conduction delay to determine a pacing cycle length (PCLlong) of each successive ‘long’ pacing pulse.

6. The method of claim 1, wherein the ventricular tachycardia is identified based at least in part on the tachycardia cycle length (TCL) and a difference in timing between two or more electrodes.

7. The method of claim 6, wherein the one or more electrodes used to delivery overdrive pacing are selected to be a ventricular electrode with the latest activation during the ventricular tachycardia.

8. The method of claim 7, wherein an algorithm for the overdrive pacing is determined based at least in part on previous attempts to terminate the ventricular tachycardia by the overdrive pacing.

9. The method of claim 1, wherein overdrive pacing comprises delivering pacing signals from an atrial electrode adjacent an atrium of the heart while ventricular overdrive pacing is also being delivered.

10. A method for terminating a ventricular tachycardia having a tachycardia cycle length (TCL), comprising:
delivering overdrive pacing including:
a) at least one pacing pulse having a pacing cycle length (PCLshort) that is less than 90% of the tachycardia cycle length (TCL);
b) followed by delivering of at least one ‘long’ pacing pulse having a pacing cycle length (PCLlong) that is greater than 90% of the tachycardia cycle length (TCL);
sensing ventricular signals during the delivery of the overdrive pacing; and
adjusting the delivery of the overdrive pacing based at least in part on the sensed ventricular signals.

11. The method of claim 10, wherein a morphology of the sensed ventricular signals is compared between the pacing pulses during the overdrive pacing to adjust the delivery of the overdrive pacing.

12. The method of claim 11, wherein adjusting the delivery of overdrive pacing includes alternating the at least one ‘long’ pacing pulse having a pacing cycle length (PCLlong) that is greater than 90% of the tachycardia cycle length (TCL) followed by at least one ‘short’ pacing pulse having a pacing cycle length (PCLshort) that is less than 90% of the tachycardia cycle length (TCL).

13. A system for delivering therapy to terminate a tachycardia event in a heart of a patient, comprising:
a pacing device configured to be implanted in the patient's body and comprising a processor;
a plurality of electrodes coupled to the processor and sized for implantation within the patient's body;
wherein the processor is configured to:
measure a time interval from stimulation from a first electrode to a sensed ventricular signal from at least one second electrode when the patient is not in ventricular tachycardia;
detect a ventricular tachycardia having a tachycardial cycle length (TCL);
deliver overdrive pacing from one or more electrodes in response to the detection;
sense ventricular signals from the at least one second electrode during the delivery of overdrive pacing; and
adjust the delivery of overdrive pacing based on the sensed ventricular signals from the at least one second electrode.

14. The system of claim 13, wherein the processor is further configured to adjust the delivery of overdrive pacing includes both adjusting timing of pacing pulses of the overdrive pacing and power output of the overdrive pacing.

15. The system of claim 14, wherein the processor is further configured to adjust the delivery of overdrive pacing by delivering alternating at least one ‘long’ pacing pulse with a pacing cycle length (PCLlong) greater than 90% of the tachycardia cycle length (TCL) of the ventricular tachycardia followed by at least one ‘short’ pacing pulse with a pacing cycle length (PCLshort) less than 90% of the tachycardia cycle length.

16. The system of claim 15, wherein the processor is further configured to repeat delivering of a ‘long’ pacing pulse followed by a ‘short’ pacing pulse one or more times, wherein each successive ‘short’ pacing pulse has a shorter pacing cycle length (PCLshort) than a previous ‘short’ pacing pulse until there is evidence of either tachycardia termination or tachycardia acceleration of the ventricular tachycardia.

17. The system of claim 15, wherein the processor is further configured to:
estimate conduction delay within a reentrant circuit causing the ventricular tachycardia during the overdrive pacing based on one or more sensed ventricular signals from the at least one second electrode; and
use the conduction delay to determine a pacing cycle length (PCLlong) of each successive ‘long’ pacing pulse.

18. The system of claim 13, wherein the processor is further configured to identify the ventricular tachycardia based at least in part on the tachycardia cycle length (TCL) and a difference in timing between two or more electrodes of the plurality of electrodes.

19. The system of claim 18, wherein the processor is further configured to select one or more electrodes to deliver overdrive pacing to be a ventricular electrode with the latest activation during the ventricular tachycardia.

20. The system of claim 19, wherein the processor is further configured to determine an algorithm for the overdrive pacing based at least in part on previous attempts to terminate the ventricular tachycardia by the overdrive pacing.

21-24. (canceled)
